SEQUENCE LISTING (7 sequences)

SEQ ID NO: 1 - protein, 361 aa, Homo sapiens
MWVLTPAAFAGKLLSVFRQPLSSLWRSLVPLFCWLRATFWLLATKRRKQQLVLRGPDETKEEEEDPPLPTTPTSVNYHFTRQCNYKCGFCFHTAKTSFVLPLEEAKRGLLLLKEAGMEKINFSGGEPFLQDRGEYLGKLVRFCKVELRLPSVSIVSNGSLIRERWFQNYGEYLDILAISCDSFDEEVNVLIGRGQGKKNHVENLQKLRRWCRDYRVAFKINSVINRFNVEEDMTEQIKALNPVRWKVFQCLLIEGENCGEDALREAERFVIGDEEFERFLERHKEVSCLVPESNQKMKDSYLILDEYMRFLNCRKGRKDPSKSILDVGVEEAIKFSGFDEKMFLKRGGKYIWSKADLKLDW

SEQ ID NO: 2 - protein, 274 aa, Selenomonas ruminantium
MAYKVNLHITQKCNYACKYCFAHFDHHNDLTLGQWKHIIDNLKTSGLVDAINFAGGEPVLHRDFAAIVNYAYDQGFKLSIITNGSLMLNPKLMPPELFAKFDTLGISVDSINPKTLIALGACNNSQEVLSYDKLSHLITLARSVNPTIRIKLNTVITNLNADEDLTIIGQELDIARWKMLRMKLFIHEGFNNAPLLVSQADFDGFVERHAEVSHDIVPENDLTRSYIMVDNQGRLLDDETEEYKVVGSLLAEDFGTVFDRYHFDEATYASRYAG

SEQ ID NO: 3 - protein, 298 aa, Psychrobacter lutiphocae
MHNHNKIANKELVVNWHITEACNYRCGYCFAKWGKQKGELIQDVASISQLMDAISGLPAVLNQMHAANFEGVRLNLVGGETFLNYRKIKEVVKQAKKRGLKLSAITNGSRINNDFINLIANNFASIGFSVDSVDNSTNLNIGRVEKNAVMNPEKIIHTIASIRAINPKIEIKVNTVVSDLNKSEDLSDFIGQVMPNKWKIFKVLPVVANHHLISEEQFTRFLRRHQRFGEIIYAEDNTEMVDSYIMIDPIGRFFQNSDFNNGYYYSRPILQVGIHQAFNEINFNANKFYSRYKRASLN

SEQ ID NO: 4 - protein, 333 aa, Fibrobacter sp. UWH6
MNIKTIVINWHITESCNYKCKYCFAKWNRVKEIWTNPDNVRKILENLKSIRLEDCLFTQKRLNIVGGEPILQQERLWQVIKMAHEMDFEISIITNGSHLEYICPFVHLISQVGVSIDSFDHKTNVRIGRECNGKTISFQQLKEKLEELRTLNPGLNIKINTVVNEYNFNEILVDRMAELKIDKWKILRQLPFDGKEGISDFKFNTFLFNNLKEEKMPKKDPLSNFLAAFSAPQKQNNVIFVEDNDVMTESYLMIAPDGRLFQNGHKEYEYSHPLTEISIDEALEEINFDQEKFNNRYENYATEEAKYRMEEFFLMNEYEDVSFDCCCPFGDKD

SEQ ID NO: 5 - protein, 308 aa, Fibrobacter sp. UWH6
MEIHMTSIQELVINFHMTEACNYRCGYCYATWQDNSSDTELHHASENIHSLLLKLADYFFADNSLRQTLKYQSVRINFAGGEPVMLGSRFIDAILFAKQLGFATSIITNGHLLSTVMLKKIAVHLDMLGISFDTGDYLIAQSIGRVDRKKSWLSPARVLDVVTQYRALNPKGKVKINTVVNAYNWRENLTQTITQLKPDKWKLLRVLPVYSKEMTVLQWQYESYVHKHQVHADVIVVEDNDDMWQSYLMINPEGRFYQNAGACKGLTYSPPVLEVGVEEALKYINFNAEAFSKRYQSIHLPLAMSAGA

SEQ ID NO: 6 - protein, 8 aa, Homo sapiens
  misc_feature (2)..(4): Xaa can be any naturally occurring amino acid
  misc_feature (6)..(7): Xaa can be any naturally occurring amino acid
CXXXCXXC

SEQ ID NO: 7 - protein, 8 aa, Homo sapiens
  misc_feature (4)..(4): Xaa can be any naturally occurring amino acid
  misc_feature (6)..(7): Xaa can be any naturally occurring amino acid
CNYXCXXC

(19) United States
(12) Patent Application Publication
(10) Pub. No.: US 2023/0138178 A1
(43) Pub. Date: May 4, 2023

(54) Methods and Compositions for Producing a Heterologous Antiviral Compound in a Host Cell

(71) Applicant: The Regents of the University of California, Oakland, CA (US)

(72) Inventors: Michael Cronce, Berkeley, CA (US); Jeffrey S. Cox, San Francisco, CA (US); Jay D. Keasling, Berkeley, CA (US)

(73) Assignee: The Regents of the University of California, Oakland, CA (US)

(21) Appl. No.: 17/849,327

(22) Filed: Jun. 24, 2022

Related U.S. Application Data
(63) Continuation-in-part of application No. PCT/US2020/066731, filed on Dec. 22, 2020.
(60) Provisional application No. 62/953,074, filed on Dec. 23, 2019.

(51) Int. Cl. A61K 35/74; A61P 31/12; C12N 15/74

(52) U.S. Cl. CPC A61K 35/74; A61P 31/12; C12N 15/74

(57) ABSTRACT

The present invention provides for a genetically modified gut bacterial cell capable of producing an antiviral compound. The genetically modified gut bacterial cell can be introduced to a subject to colonize the gastrointestinal tract of the subject, produce the antiviral compound and increase resistance to a virus infection.

### CROSS-REFERENCE TO RELATED APPLICATIONS

This application claims priority as a continuation-in-part application of PCT International Patent Application No. PCT/US2020/066731, filed Dec. 22, 2020, which in turn claims priority to U.S. Provisional Patent Application Ser. No. 62/953,074, filed Dec. 23, 2019, which is hereby incorporated by reference.

### STATEMENT OF GOVERNMENTAL SUPPORT

The invention was made with government support under Contract Nos. DE-ACO2-05CH11231 awarded by the U.S. Department of Energy, and Grant Nos. P01 AI063302, P01 AI120694, DP1 AI124619 from the National Institutes of Health. The government has certain rights in the invention.

### REFERENCES TO SUBMISSION OF A SEQUENCE LISTING

This application includes a Sequence Listing as a text file named “2019_085_03_Sequence_Listing_ST25” created Feb. 10, 2021 and containing 14,907 bytes. The material contained in this text file is incorporated by reference in its entirety for all purposes.

### FIELD OF THE INVENTION

The present invention is in the field of producing an antiviral compound in a gut bacterial cell.

### BACKGROUND OF THE INVENTION

Gizzi et al. (“A Naturally Occurring Antiviral Ribonucleotide Encoded by the Human Genome.” Nature, vol. 558, no. 7711, 2018, pp. 610-614) demonstrated Viperin, a human gene, can produce antiviral small molecules (ddhCTP) that acts as a chain terminator for RNA viruses. In the human body, Viperin is overexpressed during immune responses; this, in turn, produces ddhCTP to interfere with the virus' ability to replicate. The Gizzi et al. study was challenging because Viperin contains iron-sulfur clusters, and therefore must be maintained in anaerobic environments, and also requires iron and sulfur for proper protein folding.

### SUMMARY OF THE INVENTION

This present invention provides for a genetically modified host cell capable of producing an antiviral compound. In some embodiments, the antiviral compound is also an anti-bacterial compound.

In some embodiments, the genetically modified host cell is prokaryotic or eukaryotic cell. In some embodiments, the genetically modified host cell is commensal or non-commensal with animal. In some embodiments, the genetically modified host cell is a gut bacterial cell. In some embodiments, the host cell is microorganism, such as a bacterial cell or a yeast cell. In some embodiments, the host cell is a cell that is non-pathogenic to an animal, and/or is considered generally recognized as safe (GRAS) by the U.S. Food and Drug Administration (FDA).

This present invention provides for a genetically modified host cell comprises a nucleic acid encoding a polypeptide, or set of polypeptides, having a biological activity of synthesizing an antiviral compound operatively linked to one or more promoters capable of expressing the polypeptide(s) in the genetically modified host cell. In some embodiments, the polypeptide has an amino acid sequence that has at least 70%, 80%, 90%, 95%, 99%, or 100% amino acid residue identity with one of SEQ ID NOs:1-5 (or the amino acid sequence of any one of the viperin homologs shown in Table 1). In some embodiments, the polypeptide comprises a conserved 4Fe-4S domain (Fer4_12 (PF13353.6)) and/or a Radical SAM superfamily domain Radical_SAM (PF04055.21). In some embodiments, the conserved 4Fe-4S domain comprises conserved closely spaced cysteine residues in one of the following amino acid sequence: CXXXCXXC (SEQ ID NO:6) or CNYXCXXC (SEQ ID NO:7), In some embodiments, the polypeptide has the biological activity of synthesizing an antiviral compound, such as ddhN or ddhNTP, such as ddhA, ddhC, ddhG, ddhU, ddhATP, ddhCTP, ddhGTP, and/or ddhUTP.

In some embodiments, the polypeptide has an amino acid sequence that has at least 70%, 80%, 90%, 95%, 99%, or 100% amino acid residue identity with SEQ ID NO:1, and the conserved 4Fe-4S domain, such as CXXXCXXC (SEQ ID NO:6) or CNYXCXXC (SEQ ID NO:7), and the polypeptide has the biological activity of synthesizing ddhCTP.

In some embodiments, the polypeptide has an amino acid sequence that has at least 70%, 80%, 90%, 95%, 99%, or 100% amino acid residue identity with SEQ ID NO:2, and the conserved 4Fe-4S domain, such as CXXXCXXC (SEQ ID NO:6) or CNYXCXXC (SEQ ID NO:7), and the polypeptide has the biological activity of synthesizing ddhCTP.

In some embodiments, the polypeptide has an amino acid sequence that has at least 70%, 80%, 90%, 95%, 99%, or 100% amino acid residue identity with SEQ ID NO:3, and the conserved 4Fe-4S domain, such as CXXXCXXC (SEQ ID NO:6) or CNYXCXXC (SEQ ID NO:7), and the polypeptide has the biological activity of synthesizing ddhUTP.

In some embodiments, the polypeptide has an amino acid sequence that has at least 70%, 80%, 90%, 95%, 99%, or 100% amino acid residue identity with SEQ ID NO:4, and the conserved 4Fe-4S domain, such as CXXXCXXC (SEQ ID NO:6) or CNYXCXXC (SEQ ID NO:7), and the polypeptide has the biological activity of synthesizing ddhGTP.

In some embodiments, the polypeptide has an amino acid sequence that has at least 70%, 80%, 90%, 95%, 99%, or 100% amino acid residue identity with SEQ ID NO:5, and the conserved 4Fe-4S domain, such as CXXXCXXC (SEQ ID αN0:6) or CNYXCXXC (SEQ ID NO:7), and the polypeptide has the biological activity of synthesizing ddhATP, ddhCTP, ddhGTP, and/or ddhUTP.

In some embodiments, the polypeptide has an amino acid sequence that comprises amino acid residues that are conserved among SEQ ID NOs:1-5. In some embodiments, the conserved amino acid residues correspond to the domains indicated herein. In some embodiments, the polypeptide has an amino acid sequence that comprises a N-terminal domain comprising an amphipathic alpha-helix (corresponding to positions 9-42 of SEQ ID NO:1), a central terminal domain comprising a radical SAM (corresponding to positions 71-182 of SEQ ID NO:1), and a C-terminal domain (corresponding to positions 218-361 of SEQ ID NO:1). The amphipathic alpha-helix mediates localization to the ER and LDs, induces crystalloid ER and morphological changes when overexpressed, inhibition of soluble protein secretion, binding to the Fe/S assembly factor CIA2A, and/or required for antiviral activity against some viruses. The radical SAM harbors SAM-binding domain, Fe—S cluster binding, enzymativ activity, binding to immune factors, proper protein folding, and/or required for antiviral; activity against several viruses. The C-terminal domain is required for oligomerization, binding to the Fe/S assembly factors CIA2B and MMS19, binding to HCV NS5A and to DENV-2 and ZIKV NS3, and/or required for antiviral activity against some flaviviruses.

This present invention provides for a genetically modified gut bacterial cell comprises a nucleic acid encoding a polypeptide, or set of polypeptides, having a biological activity of synthesizing an antiviral compound operatively linked to one or more promoters capable of expressing the polypeptide(s) in the genetically modified gut bacterial cell. In some embodiments, the genetically modified gut bacterial cell is a gut commensal bacterium. In some embodiments, the genetically modified gut bacterial cell is a Bacteroides cell. In some embodiments, the Bacteroides cell is a Bacteroides thetaiotaomicron, Bacteroides plebus or Bacteroides ovatus cell.

In some embodiments, the polypeptide is a naturally occurring or synthetically modified polypeptide. In some embodiments, the polypeptide is a human polypeptide. In some embodiments, the antiviral compound is capable of terminating DNA or RNA virus replication. In some embodiments, the antiviral compound capable of terminating DNA or RNA virus replication is a nucleotide. In some embodiments, the antiviral compound capable of terminating DNA or RNA virus replication is ddhCTP. In some embodiments, the DNA virus has single-stranded DNA or doube-stranded DNA. In some embodiments, the polypeptide (or viperin or SEQ ID NO:1-5) is heterologous to the genetically modified gut bacterial cell and/or the promoter. In some embodiments, the antiviral compound is ddhCTP, and the polypeptide(s) is Viperin, and optionally Cytidine/Uridine Monophosphate Kinase 2 (CMPK2). CMPK2 produces more substrate for the Viperin to produce ddhCTP. In some embodiments, the Viperin is human Viperin. In some embodiments, the CMPK2 is human CMPK2. In some embodiments, the Viperin is truncated to remove the N-terminal human localization signal. In some embodiments, the nucleic acid is a high expression vector capable of overexpression of the polypeptide(s). In some embodiments, the polypeptide comprises a N-terminal tag that increases the expression and/or solubility of the polypeptide, such as MBP-GGGS-(E. coli), NusA-GGGS-(B. thetaiotaomicron), SUMO-GGGS-(Saccharomyces cerevisiae), or the like. In some embodiments, the N-terminal tag is homologous to a Bacteroides cell or the genetically modified gut bacterial cell. In some embodiments, the N-terminal tag is heterologous to a Bacteroides cell or the genetically modified gut bacterial cell.

In some embodiments, the promoter is constitutive. In some embodiments, the promoter is inducible. In some embodiments, the promoter is induced by a signal. In some embodiments, the signal is a signal produced by a diet, or a signal produced by the presence of a pathogen. In some embodiments, the pathogen is a virus. In some embodiments, the virus is a DNA or RNA virus.

Viperin has been shown to inhibit viral infection, such as infections by lymphocytic choriomeningitis virus (LCMV), West Nile Virus (WNV), Dengue Virus, Hepatitis C Virus (HCV), Chikungunya Virus, Human Immunodeficiency Virus (HIV), Cytomegalovirus, Zika Virus, Enterovirus A71, and the like, including other viruses described herein. LCMV is a member of the Arenaviridae family of viruses, which are single-stranded RNA viruses. WNV is a member of the Flaviviridae family of viruses, specifically from the genus Elavivirus, which are single-stranded RNA viruses. Dengue Virus is a member of the Flaviviridae family of viruses, specifically from the genus Flavivirus, and is a positive-sense single-stranded RNA virus. HCV is a member of the family of Flaviviridae viruses, specifically from the genus Hepacivirus, and is a positive-sense single-stranded RNA virus. Chikungunya Virus is a member of the Togaviridae family of viruses, specifically from the genus Alphavirus, and is a positive-sense single-stranded RNA virus. HIV is a member of the Retroviridae family of viruses, specifically from the genus Lentivirus, and is a positive-sense single-stranded RNA virus. In some embodiments, the virus is a virus that infects a mammal, such as a human or a domesticated mammal, such as a dog, cat, cattle, horse, sheep, or goat, or the like.

Viperin has been shown to also inhibit bacterial infection by a pathogenic bacterium, such as infections by a Mycobacterium cell, such as Mycobacterium tuberculosis. In some embodiments, the pathogenic bacterium is a bacterium that infects a mammal, such as a human or a domesticated mammal, such as a dog, cat, cattle, horse, sheep, or goat, or the like.

In some embodiments, the genetically modified host cell is capable of colonizing an animal gastrointestinal (GI) tract. In some embodiments, the genetically modified gut bacterial cell is capable of colonizing an animal gastrointestinal (GI) tract. In some embodiments, the animal is a mammal, or a domesticated mammal, such as a dog, cat, cattle, horse, sheep, or goat, or the like. In some embodiments, the mammal is a primate. In some embodiments, the primate is a human. In some embodiments, the mammal is a rodent. In some embodiments, the rodent is a mouse, rat, or rabbit. In some embodiments, when the genetically modified gut bacterial cell is colonizing the GI tract of an animal, the antiviral compound produced confers resistance or suppression of an RNA virus, and/or an infection thereof, in the animal and/or the GI tract of the animal.

In some embodiments, the genetically modified host cell further comprises the polypeptide(s). In some embodiments, the genetically modified host cell further comprises the antiviral compound. In some embodiments, the genetically modified gut bacterial cell further comprises the polypeptide(s). In some embodiments, the genetically modified gut bacterial cell further comprises the antiviral compound.

The present invention provides for a composition comprising the genetically modified host cell of the present invention, and the antiviral compound, produced by the genetically modified host cell, in the composition but outside the genetically modified host cell; in that the antiviral compounds are produced by the genetically modified host cell and transported, moved, released or diffused to the outside of the genetically modified host cell.

The present invention provides for a composition comprising the genetically modified gut bacterial cell of the present invention, and the antiviral compound, produced by the genetically modified gut bacterial cell, in the composition but outside the genetically modified gut bacterial cell; in that the antiviral compounds are produced by the genetically modified gut bacterial cell and transported, moved, released or diffused to the outside of the genetically modified gut bacterial cell.

The present invention provides for an antiviral compound produced by the genetically modified host cell of the present invention. The present invention provides for an antiviral compound produced by the genetically modified gut bacterial cell of the present invention. In some embodiments, the antiviral compound is a small molecule. In some embodiments, the antiviral compound is a nucleotide derivative.

The present invention provides for a non-human animal comprising the genetically modified gut bacterial cell of the present invention in the gastrointestinal (GI) tract of the non-human animal.

The present invention provides for a method for making the genetically modified host cell of the present invention, the method comprising: (a) optionally constructing a nucleic acid encoding a polypeptide, or set of polypeptides, having a biological activity of synthesizing an antiviral compound operatively linked to one or more promoters capable of expressing the polypeptide(s) in a genetically modified host cell, (b) introducing the nucleic acid into a host cell to generate the genetically modified host cell of the present invention, (c) optionally expressing the polypeptide(s) in order to produce the antiviral compound, and (d) optionally separating or isolating or purifying the antiviral compound from the genetically modified host cell.

The present invention provides for a method for producing an antiviral compound in an animal, the method comprising: (a) optionally constructing a nucleic acid encoding a polypeptide, or set of polypeptides, having a biological activity of synthesizing an antiviral compound operatively linked to one or more promoters capable of expressing the polypeptide(s) in a genetically modified gut bacterial cell, (b) optionally introducing the nucleic acid into a gut bacterial cell to generate the genetically modified gut bacterial cell of the present invention, (c) introducing the genetically modified gut bacterial cell of the present invention into a gastrointestinal (GI) tract of an animal, and (d) producing the antiviral compound in the GI tract, wherein the genetically modified gut bacterial cell expresses the polypeptide(s) which in turn produces the antiviral compound in the genetically modified gut bacterial cell, such that the antiviral compound is transported, moved, released or diffused to the outside of the genetically modified gut bacterial cell into the GI tract. In some embodiments, the animal is increased in its resistance to a virus.

The present invention provides for a method for reducing the likelihood of a virus infection or reducing the severity or curing a virus infection in a subject, the method comprising: (a) administering a therapeutically sufficient number of a genetically modified gut bacterial cell of the present invention to a subject in need of such treatment, wherein the likelihood of a virus infection in the subject is reduced, the severity of a virus infection is reduced in the subject or the subject is cured of a virus infection.

In some embodiments, the subject has, or is suspected to have, an increased risk to the virus infection, or is, or is suspected to be, infected with the virus, or is diagnosed with the virus infection.

The present invention provides for: 3′-deoxy-3′, 4′-didehydro-CTP, or ddhCTP, is an inducible ribonucleic chain terminator produced by VIPERIN, a protein reported to suppress a variety of RNA viruses, including Dengue, Influenza A, Chikungunya, Human Cytomegalovirus, Zika, West Nile, Hepatitis C, Enterovirus A71, and Human Immunodeficiency Virus (HIV). Importantly, because the nucleoside form of ddhCTP penetrates cell and is naturally converted into its active form via phosphorylation, this molecule is a candidate as a naturally-derived therapeutic that may broadly increase viral resistance in humans. VIPERIN may also mediate resistance to intracellular bacterial pathogens, indicating that ddhCTP may also function to promote host responses to combat a wide array of infectious agents. Preliminary data show that chemically synthesized ddhC can modulate innate immune signaling and lead to restriction of the important human pathogen Mycobacterium tuberculosis. It is demonstrate ddhCTP holds broader biological activity than previously reported and that ddhNTP compound family produced by VIPERIN homologs serves as potent antiinfective small molecules capable of suppressing bacterial and viral infections, such as from Mycobacterium tuberculosis and SARS-COV-2 infection.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows an embodiment of the invention.

FIG. 2 shows a nucleic acid construct used for the validation of pNBU2 integration and reporter activity.

FIG. 3 shows the gel results for validation of pNBU2 integration.

FIG. 4 shows the reporter activity using various promoters.

FIG. 5 shows the biological activity of Viperin (Ng and Hiscox, Cell Host & Microbe, Volume 24, Issue 2, pp. 181-183 (2018)).

FIG. 6 shows the HPLC results that show B. thetaiotaomicron produces ddhCTP in vivo.

FIG. 7A shows in vivo ddhCTP production and ddhCTP LC-MS confirmation.

FIG. 7B shows in vivo 5′-dA production in strains expressing CMPK2 and VIPERIN.

FIG. 7C shows in vivo detected protein coverage of solubility tagged Viperin using shotgun proteomics.

FIG. 8 shows the ddhCTP peak identified using an orthogonal analytical approach.

FIG. 9 shows the structure of vector pMJC28-2.

FIG. 10A shows transcriptional profiling of BMDMs. ddhC-mediated transcriptional changes.

FIG. 10B shows transcriptional profiling of BMDMs. Viperin expression levels following Poly(I:C) treatment or ddhC-Poly(I:C) co-stimulation.

FIG. 11A. Effect of ddhC on the replication of HSV-1. ddhC increases replication of HSV-1, a dsDNA virus.

FIG. 11B. Effect of ddhC on the replication of Vaccinia virus.

FIG. 11C. Effect of ddhC on the replication of LCMV. ddhC suppresses replication of LCMV, a negative strand RNA virus.

FIG. 12A. Effect of ddhNTPs on the replication of Zika Virus.

FIG. 12B. Effect of ddhNTPs on the replication of Coxsackie B4 Virus.

FIG. 12C. Effect of ddhNTPs on the replication of Influenza A.

FIG. 12D. Effect of ddhNTPs on the replication of Enterovirus A-71.

FIG. 12E. Effect of ddhNTPs on the replication of SARS-CoV.

FIG. 13. To determine the extent to which other ddhN derivatives synergize with TLR agonists to enhance NFkB signaling, RAW264.7 cells containing an NFkB reporter are pre-treated overnight with either ddhC or ddhG and subsequently stimulated with 500 ng/mL LPS, 1 μg/mL PAM, or 1 μg/mL R848. The results show that both ddhC and ddhG agonize TLR-mediated NFkB activation.

FIG. 14. To determine whether ddhNTPs exhibit cytotoxic effects, Vero cells are treated overnight with increasing doses of ddhA, ddhC, or ddhG.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “cell”, “host cell” and “host microorganism” are used interchangeably herein to refer to a living biological cell that can be transformed via insertion of an expression vector.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host microorganism, thereby causing the cell to express nucleic acids and/or proteins other than those native to the cell, or in a manner not native to the cell. An “expression vector” contains a sequence of nucleic acids (ordinarily RNA or DNA) to be expressed by the host microorganism. Optionally, the expression vector also comprises materials to aid in achieving entry of the nucleic acid into the host microorganism, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host microorganism and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to an organism or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

This invention can be as a prophylactic antiviral for travelers, military personnel, or those prone to viral infections. The user would colonize their GI tract with this microbe. Over time, this strain would produce ddhCTP to prophylactically suppress RNA viruses as they entered the body. The best use of the invention would be for those at high risk for contracting viral infections or for situations in which there is high risk for contracting viral infections.

This strain could also be used for industrial production of ddhCTP, and other nucleoside derivatives, using B. thetaiotaomicron as a chassis. In some embodiments, the bacteroides can produce ddhCTP in vivo without need for any auxiliary plasmid(s), such as comprising any exogenous iron-sulfur cluster producing genetic elements.

The amino acid sequence of viperin is as follows:

```
(SEQ ID NO: 1)
                10 20 30
MWVLTPAAFA GKLLSVFRQP LSSLWRSLVP
                40 50 60
LFCWLRATFW LLATKRRKQQ LVLRGPDETK
                70 80 90
EEEEDPPLPT TPTSVNYHFT RQCNYKCGFC
              100 110 120
FHTAKTSFVL PLEEAKRGLL LLKEAGMEKI
              130 140 150
NFSGGEPFLQ DRGEYLGKLV RFCKVELRLP
              160 170 180
SVSIVSNGSL IRERWFQNYG EYLDILAISC
              190 200 210
DSFDEEVNVL IGRGQGKKNH VENLQKLRRW
              220 230 240
CRDYRVAFKI NSVINRFNVE EDMTEQIKAL
              250 260 270
NPVRWKVFQC LLIEGENCGE DALREAERFV
              280 290 300
IGDEEEERFL ERHKEVSCLV PESNQKMKDS
              310 320 330
YLILDEYMRF LNCRKGRKDP SKSILDVGVE
              340 350 360
EAIKESGFDE KMFLKRGGKY IWSKADLKLD
W
```

The following protein sequences produce the indicated ddhNs (Bernheim, Aude, et al. “Prokaryotic Viperins Produce Diverse Antiviral Molecules.” Nature, 2020). Also included is a sequence that produces a mixture of all three ddhUTP, ddhCTP, and ddhGTP from a single enzyme. This promiscuity suggests it may also produce ddhATP.

The Selenomonas rurninatiurn S137 viperin homolog produces ddhCTP. The amino acid sequence of Selenomonas ruminatium S137 viperin homolog is as follows:

(SEQ ID NO: 2)
MAYKVNLHITQKCNYACKYCFAHFDHHNDLTLGQW
KHIIDNLKTSGLVDAINFAGGEPVLHRDEAAIVNY
AYDQGFKLSIITNGSLMLNPKLMPPELFAKFDTLG
ISVDSINPKTLIALGACNNSQEVLSYDKLSHLITL
ARSVNPTIRIKLNTVITNLNADEDLTIIGQELDIA
RWKMLRMKLFIHEGFNNAPLLVSQADFDGFVERHA
EVSHDIVPENDLTRSYIMVDNQGRLLDDETEEYKV
VGSLLAEDFGTVFDRYHFDEATYASRYAG

The Psychrobacter lutiphocae DSM 21542 viperin homolog produces ddhUTP. The amino acid sequence of Psychrobacter lutiphocae DSM 21542 viperin homolog is as follows:

(SEQ ID NO: 3)
MHNHNKIANKELVVNWHITEACNYRCGYCFAKWGK
QKGELIQDVASISQLMDAISGLPAVLNQMHAANFE
GVRLNLVGGETFLNYRKIKEVVKQAKKRG
LKLSAITNGSRINNDFINLIANNFASIGFSVDSVD
NSTNLNIGRVEKNAVMNPEKIIHTIASIRAINPKI
EIKVNTVVSDLNKSEDLSDFIGQVMPNKWKIFKVL
PVVANHHLISEEQFTRFLRRHQRFGEIIYAEDNTE
MVDSYIMIDPIGRFFQNSDFNNGYYYSRPILQVGI
HQAFNEINFNANKFYSRYKRASLN

The Fibrobacter sp. UWH6 viperin homolog produces ddhGTP. The amino acid sequence of Fibrobacter sp. UWH6 viperin homolog is as follows:

(SEQ ID NO: 4)
MNIKTIVINWHITESCNYKCKYCFAKWNRVKEIWT
NPDNVRKILENLKSIRLEDCLFTQKRLNIVGGEPI
LQQERLWQVIKMAHEMDFEISIITNGSHLEYICPF
VHLISQVGVSIDSFDHKTNVRIGRECNGKTISFQQ
LKEKLEELRTLNPGLNIKINTVVNEYNFNEILVDR
MAELKIDKWKILRQLPFDGKEGISDFKFNTFLFNN
LKEEKMPKKDPLSNFLAAFSAPQKQNNVIFVEDND
VMTESYLMIAPDGRLFQNGHKEYEYSHPLTEISID
EALEEINFDQEKFNNRYENYATEEAKYRMEEFFLM
NEYEDVSFDCCCPFGDKD

The Fibrobacter sp. UWH6 viperin homolog produces ddhCTP, ddhGTP, and ddhUTP (and possibly ddhATP). The amino acid sequence of Fibrobacter sp. UWH6 viperin homolog is as follows:

SEQ ID NO: 5)
MEIHMTSIQELVINFHMTEACNYRCGYCYATWQDN
SSDTELHHASENIHSLLLKLADYFFADNSLRQTLK
YQSVRINFAGGEPVMLGSRFIDAILFAKQLGFATS
IITNGHLLSTVMLKKIAVHLDMLGISFDTGDYLIA
QSIGRVDRKKSWLSPARVLDVVTQYRALNPKGKVK
INTVVNAYNWRENLTQTITQLKPDKWKLLRVLPVY
SKEMTVLQWQYESYVHKHQVHADVIVVEDNDDMWQ
SYLMINPEGRFYQNAGACKGLTYSPPVLEVGVEEA
LKYINFNAEAFSKRYQSIHLPLAMSAGA

Using the Fibrobacter sequence indicated above a HMMER3 model is constructed that identifies conserved structural elements of the Viperin homologs. This model revealed a highly conserved 4Fe-4S domain (Fer4_12 (PF13353.6)) and a Radical SAM superfamily domain Radical_SAM (PF04055.21). The conserved 4Fe-4S domain comprises conserved closely spaced cysteine residues in the following amino acid sequence: CXXXCXXC (SEQ ID NO:6) or CNYXCXXC (SEQ ID NO:7), These domains were also observed when constructing HMMER3 models with the other sequences listed above. Other known viperin homologs are shown in Table 1.

TABLE 1
List of viperin and homologs thereof.

| Protein | Accession No. | Organism |
|---|---|---|
| Viperin | XP_024064957.1 | Terrapene mexicana triunguis |
| Viperin | PKC63257.1 | Rhizophagus irregularis |
| Viperin | KIM76756.1 | Piloderma croceum F 1598 |
| Viperin | PNP59997.1 | Trichoderma harzianum |
| Viperin | XP_851276.1 | Canis lupus familiaris |
| Viperin | XP_001510936.1 | Ornithorhynchus anatinus |
| Viperin | KFP16729.1 | Egretta garzetta |
| Viperin | XP_006108914.2 | Myotis lucifugus |
| Viperin | ALT07788.1 | Crassostrea gigas |
| Viperin | NP_542388.2 | Homo sapiens |

Prior to this present invention, there has been no precedence for a gut commensal microbe capable of making small molecules to suppress human viral infection and none has been engineered to do so. Additionally, Bacteroides strains, such as B. thetaiotaomicron, are one of the most robust constituents of the gut microbiome, both in terms of relative abundance within the microbiome and its prevalence between people. This is important when considering which host can produce enough compound to provide a therapeutic benefit.

One can modify the expression of a gene encoding any of the enzymes taught herein by a variety of methods in accordance with the methods of the invention. Those skilled in the art would recognize that increasing gene copy number, ribosome binding site strength, promoter strength, and various transcriptional regulators can be employed to alter an enzyme expression level.

Nucleotides are crucial biomolecules for sustaining cellular life. In addition to serving as the building blocks of nucleic acids, these compounds serve a myriad of functions, such as being the primary energy donor for cellular processes, acting as organic cofactors for proteins, allosteric regulators of protein function, and functioning as metabolic activators of lipid and polysaccharide precursors Compellingly, one report showed that CDNs produced from bacterial homologs were able to modulate the mammalian innate immune response and this host response was specific to the bacterial CDN chemical identity (Whiteley et al., 2019). Since then, metabolic pathways producing novel nucleotide derivatives have been discovered in both prokaryotes and eukaryotes. This broadening class of natural products, and the enzymes that produce them, offer a new frontier of previously unknown cellular functions and new bioproducts that may be of interest to society-at large.

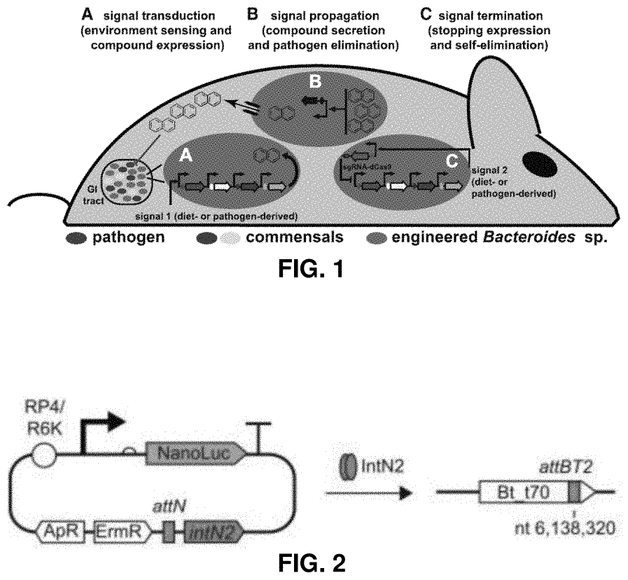

Recently, a new nucleotide analog, 3′-deoxy-3′, 4′-didehydro-CTP (ddhCTP), has been identified in mammalian cells. ddhCTP production is catalyzed by Viperin, a protein with broad spectrum antiviral properties. Originally discovered in 2001, VIPERIN has been shown to suppress a variety of RNA viruses, including Dengue, Influenza A, Chikungunya, Human Cytomegalovirus, Zika, West Nile, Hepatitis C, Enterovirus A71, and Human Immunodeficiency Virus (HIV), but its mode of action remained mysterious (Ng and Hiscox, 2018; Gizzi et al., 2018; Wei et al., 2018). Viperin was only recently found to mediate its antiviral activity via ddhCTP inhibition of RNA synthesis by directly terminating chain elongation (FIG. 1) (Gizzi et al., 2018). This work demonstrates provides an example of a human protein that directly inhibits RNA virus genome replication. Importantly, endogenously produced and exogenously administered ddhCTP/ddhC restricts flavivirus infection with minimal evidence of toxicity to the host (Gizzi et al., 2018). Surprisingly, VIPERIN may also mediate resistance to intracellular bacterial pathogens, indicating that ddhCTP may also function to promote host responses to combat a wide array of infectious agents (Helbig et al., 2019). However, no published study has evaluated the host-directed effects of this compound on mammalian cell physiology or its ability to suppress either nonflavivirus viral replication, including Filoviruses, Picornaviruses, Paramyxoviruses, Orthomyxoviruses, Coronaviruses, or bacterial pathogen growth, such as M. tuberculosis.

Preliminary data indicates ddhC, the bioavailable form of ddhCTP, stimulation modulates Toll like receptor (TLR)-mediated Type 1 interferon signaling and NFkB signaling in macrophages (FIG. 2A,B,C). Additionally, it is found that ddhC-stimulated macrophages restrict M. tuberculosis growth greater than those treated with Vehicle or Cytidine, a compound with similar chemical identity to that of ddhC (FIG. 2D). These results support a recent report revealing VIPERIN expression restricts Shigella and L. monogenes infection in a SAM-dependent manner (Helbig et al., 2019). Taken together, these data suggest mammalian cells can detect and respond to intracellular ddhCTP pools and these responses have functional consequences for host defense against a wide range of pathogens.

To determine the functional consequence of ddhC stimulation for viruses not expected to be targeted by ddhCTP, mouse macrophages are pretreated with 1 mM ddhC for 24 hours and are subsequently infected with GFP-tagged Vaccinia (Pox) virus or Herpes Simplex Virus 1 for 24 hours. Mouse macrophages are then analyzed via flow cytometry to determine the percent infectivity per well. Results indicate ddhC pretreatment had negligible effects on Vaccinia virus infection, while increasing the HSV-1 infectivity. Thus, preliminary results indicate ddhCTP suppresses TLR4-mediated Type 1 interferon induction and increases HSV-1 infectivity. Furthermore, preliminary data show the virus-directed and host-targeting (immunomodulatory) effects of ddhCTP are similar to that of Zalcitabine(ddC) (West et al, 2015), a first generation anti-HIV medication with similar chemical identity to that of ddhC. Recent reports also demonstrate that RNA-dependent RNA polymerase inhibitors known to target flaviviruses and HIV are shown to effectively incorporate into elongating RNA strands mediated by SARS-CoV-2 RNA Dependent RNA Polymerase activity (Chien et al., 2020).

Surprisingly, Viperin is evolutionarily conserved across all domains of life, suggesting a deep evolutionary history of 3′-deoxy-3′, 4′-didehydro-nucleotide triphosphates, or ddhNTPs, in biology. However, the biochemical activity of these homologs remains poorly characterized. One recent study identified a Radical SAM enzyme from Thielavia terrestris, a thermophilic fungus, as a Viperin homolog that can act on CTP, UTP, and 5-bromo-UTP to produce ddhCTP, ddhUTP, and 5-bromo-ddhUTP in vitro (Ebrahimi et al., 2020).

Taken together, these initial reports indicate that ddhCTP is a bioactive small molecule capable of suppressing RNA viruses and there exist Viperin homologs that produce a broader diversity of nucleotide analogues. It is herein demonstrated ddhCTP holds broader biological activity than previously reported and that ddhNTP compound family produced by VIPERIN homologs can serve as potent anti-infective small molecules capable of suppressing M.tb and SARS-COV-2 infection.

One advantage of the proposed technology is the ability to efficiently make anti-infective small molecules via biocatalytic approaches. Comparable production methods using synthetic chemistry are much less efficient to the point that it makes industrial scaling impractical. Additionally, the ddhNTP compound family is significantly less explored than other compound families offering new opportunities for making novel compounds with unexplored utility.

Results suggest the ddhNTP compound family produced by VIPERIN homologs can serve as potent anti-infective small molecules capable of suppressing M.tb and SARS-COV-2 infection.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### EXAMPLE 1

Viperin is expressed in bacteroides, both due to its anaerobic requirement and iron-sulfur cluster-rich metabolism. To accomplish this, a high expression vector is constructed based upon the works of Mimee et al. (2015) and Whitaker et al. (2017). Next the N-terminus of viperin is truncated to remove the human localization signal and co-expressed it with another human protein, CMPK2, to make more substrate for Viperin to use. The sequences for both of these genes are codon optimized for expression in bacteroides. HPLC-UV Vis and High Resolution LC-MS reveal that ddhCTP is only detected in microbes expressing both CMPK2 and Viperin.

This work represents the first time human proteins are heterologously expressed to make antiviral compounds in microbes. Gizzi, et al. (2018) showed only the production of ddhCTP in human cells or synthesized using cell-free systems.

FIG. 1 shows a scheme for a commensal bacteria to produce a bioactive molecule.

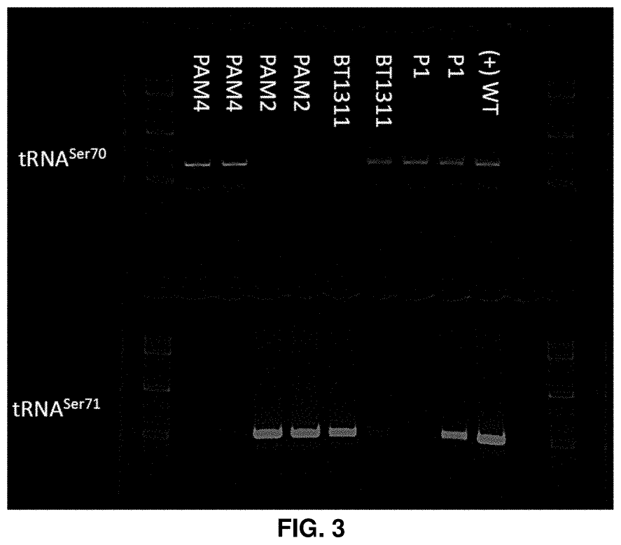

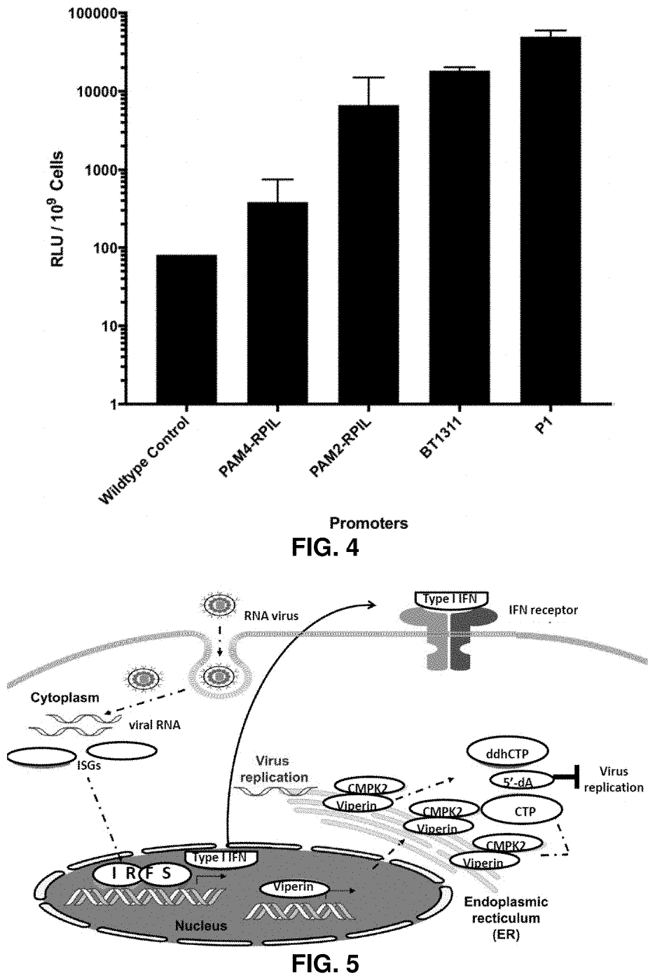

FIGS. 2-4 show the validation of pNBU2 integration and reporter activity.

FIG. 5 shows the biological activity of Viperin (Ng and Hiscox, Cell Host & Microbe, Volume 24, Issue 2, pp. 181-183 (2018)). Viperin has been shown to inhibit viral infection, such as infections by lymphocytic choriomeningitis virus (LCMV), West Nile Virus (WNV), Dengue Virus, Hepatitis C Virus (HCV), Chikungunya Virus, Human Immunodeficiency Virus (HIV), and the like. LCMV is a member of the Arenaviridae family of viruses, which are single-stranded RNA viruses. WNV is a member of the Flaviviridae family of viruses, specifically from the genus Flavivirus, which are single-stranded RNA viruses. Dengue Virus is a member of the Flaviviridae family of viruses, specifically from the genus Flavivirus, and is a positive-sense single-stranded RNA virus. HCV is a member of the family of Flaviviridae viruses, specifically from the genus Hepacivirus, and is a positive-sense single-stranded RNA virus. Chikungunya Virus is a member of the Togaviridae family of viruses, specifically from the genus Alphavirus, and is a positive-sense single-stranded RNA virus. HIV is a member of the Retroviridae family of viruses, specifically from the genus Lentivirus, and is a positive-sense single-stranded RNA virus.

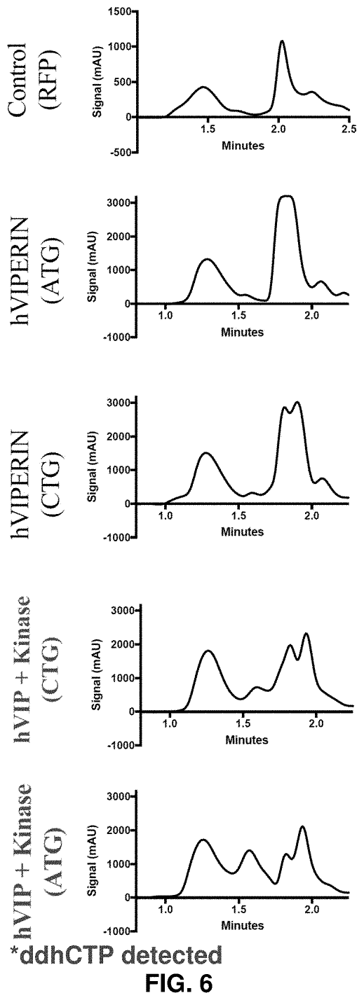

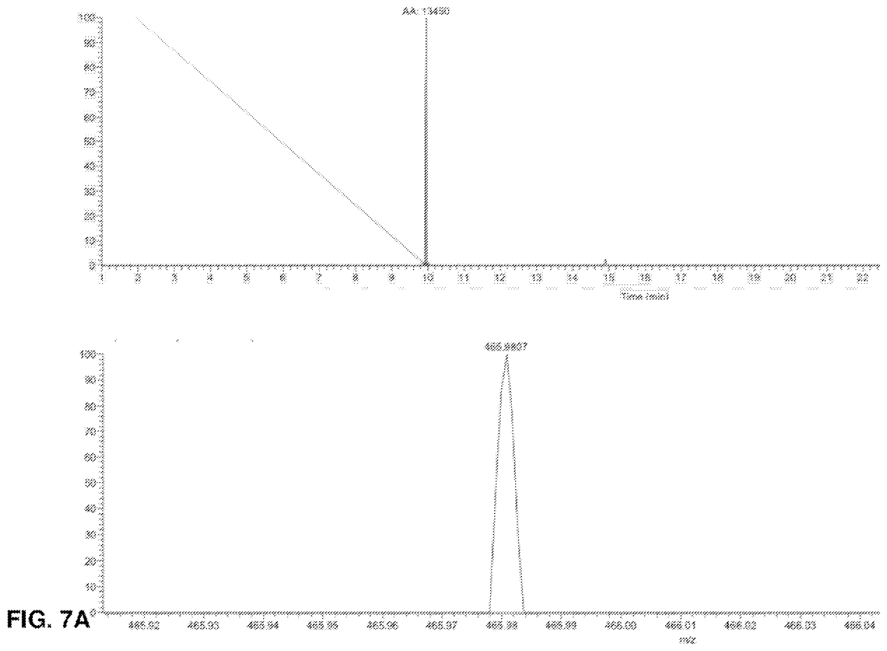

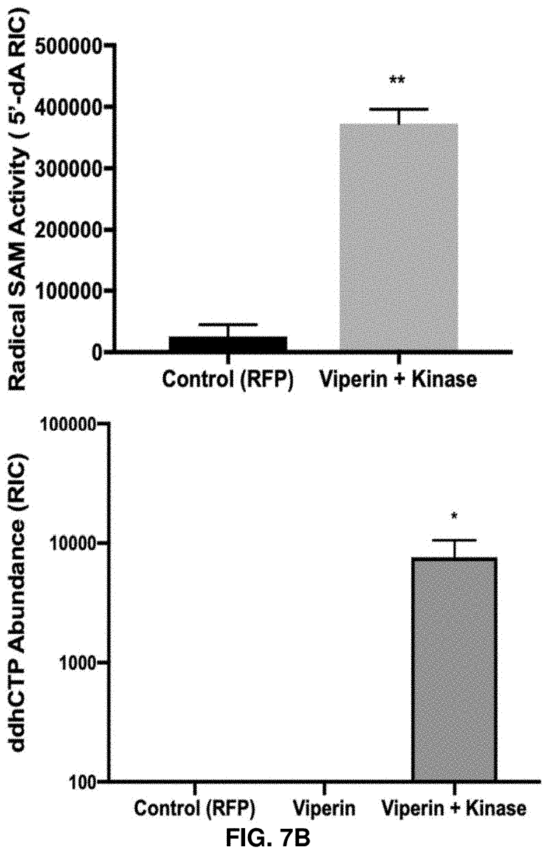

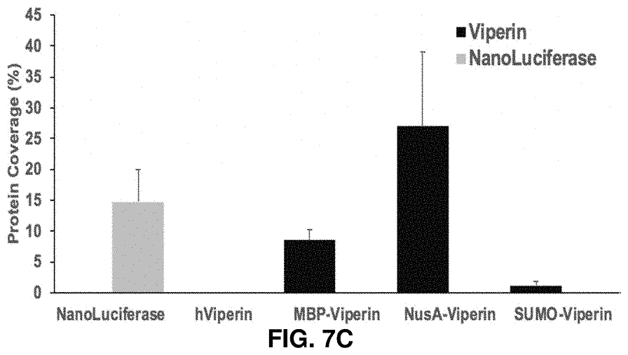

FIGS. 6, 7A, and 7B depict the HPLC and LC-MS results that show B. thetaiotaomicron produces ddhCTP in vivo.

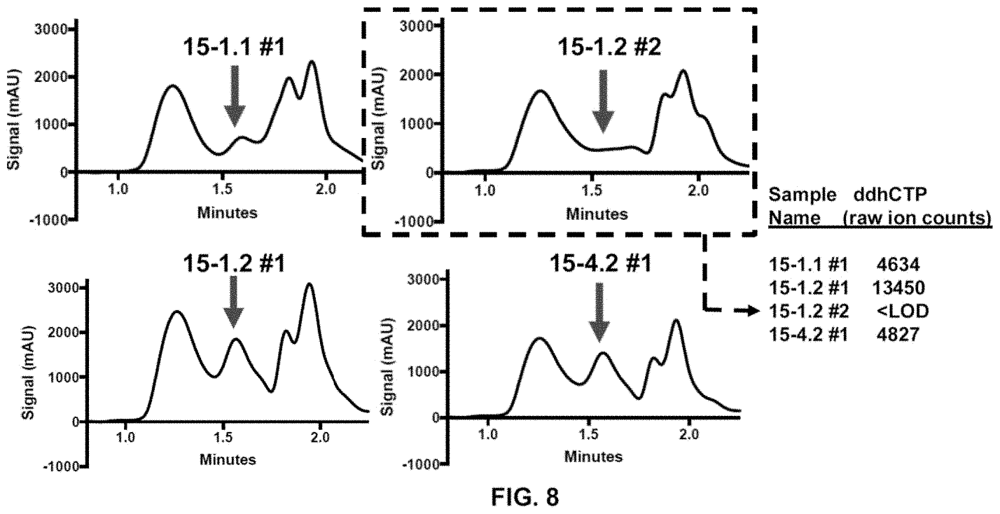

FIG. 8 shows the ddhCTP peak identified using an orthogonal analytical approach.

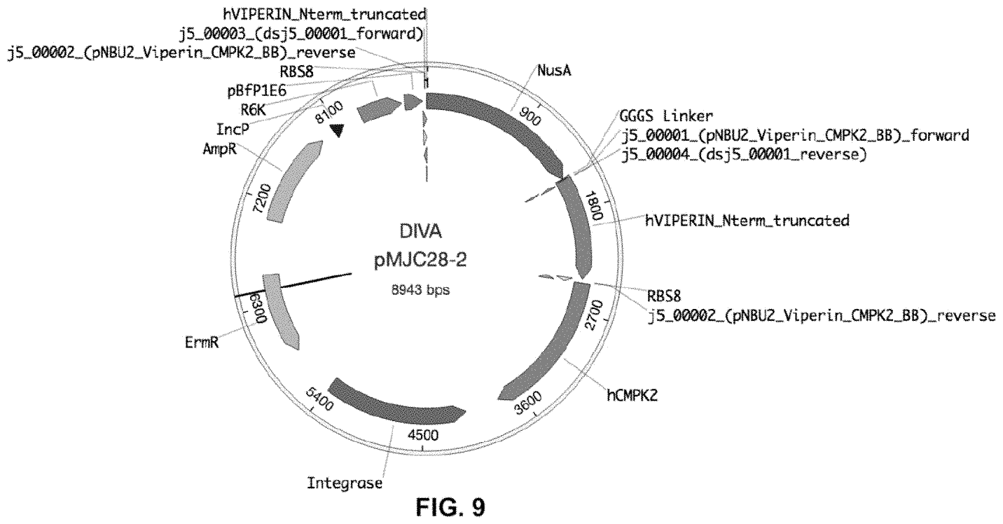

FIGS. 7C and 9 show the solubility tagged fusion protein increases peptide abundance in bacteroides. The tags synthesized can be MBP-GGGS-(E. coli), NusA-GGGS-(B. thetaiotaomicron), SUMO-GGGS-(Saccharomyces cerevisiae), or the like. The truncated viperin protein that produced compound in FIGS. 6, 7A, 7B, and 8 could not be detected via proteomics. When the truncated Viperin protein is fused to tags known to increase protein expression and/or solubility, the Viperin peptides are detected for all three tagged proteins. Interestingly, the NusA-Viperin fusion protein exhibits the greatest abundance. NusA is the only solubility tag tested that also has a homolog in bacteroides.

### EXAMPLE 2

Viperin is an Fe—S cluster containing protein, and thus, denatures under aerobic conditions. Therefore, it is hypothesized that Bacteroides thetaiotaomicron, an obligate commensal anaerobe with an Fe—S cluster rich metabolism, is a suitable host for expressing functional VIPERIN protein. Recently, numerous groups developed stably integrated expression systems in B. thetaiotaomicron^{9,17}. A luciferase expression library is constructed using previously characterized promoters and this promoter library is integrated into B. thetaiotaomicron. Luciferase expression levels agree with previously reported values (data not shown). Next, this toolkit is used to express a two-gene operon consisting of (1) a truncated, codon-optimized coding sequence of human VIPERIN and (2) CMPK2, a cytidylate (CMP) kinase found in synteny with Viperin; CMPK2 functions to ensure Viperin is not substrate-limited³. LC-MS analysis reveal detectable levels of ddhCTP and 5′-deoxyadenosine, a byproduct of SAM radical enzyme activity, only in strains co-expressing VIPERIN and CMPK2 (FIG. 7A, 7B) (N=3 per condition). In these strains, VIPERIN is undetectable via shotgun proteomics, suggesting this protein is not expressed at substantial levels. To increase protein stability, the truncated VIPERIN is fused to one of three different solubility tags (FIG. 7C). Proteomics revealed NusA and MBP greatly enhanced VIPERIN protein levels (N=3 per condition).

To determine the operon structure exhibiting the greatest ddhCTP production rate, VIPERIN and CMPK2 are fused to either NusA or MBP. Next, these fusion proteins are mixed and matched to make a combinatorial library of 5 different operons. All of the above mentioned constructs are stably integrated into B. thetaiotaomicron. Pellets, supernatant, and glycerol stocks have been prepped for all strains (N=6). Further analysis can be carried out using metabolite-targeted LC-MS and proteomic analysis.

One previous study reported ddhCTP can suppress ZIKV, HCV, WNV, and DV while having no cytotoxic effects on host cell physiology³. Additionally, other groups have shown Viperin exhibits antiviral activity beyond the Flaviviridae family of viruses. In fact, one recent study found overexpres sing the C-terminal domain of Viperin can partially suppress Enterovirus A71 infection. Whether this broader antiviral activity can be specifically ascribed to ddhCTP pharmacoactivity remains unclear.

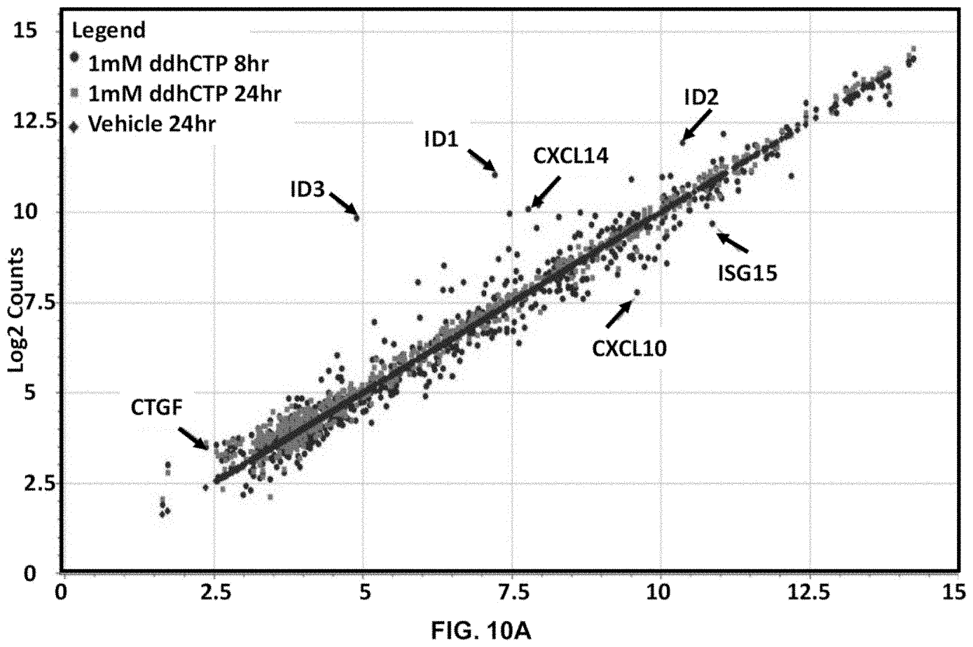

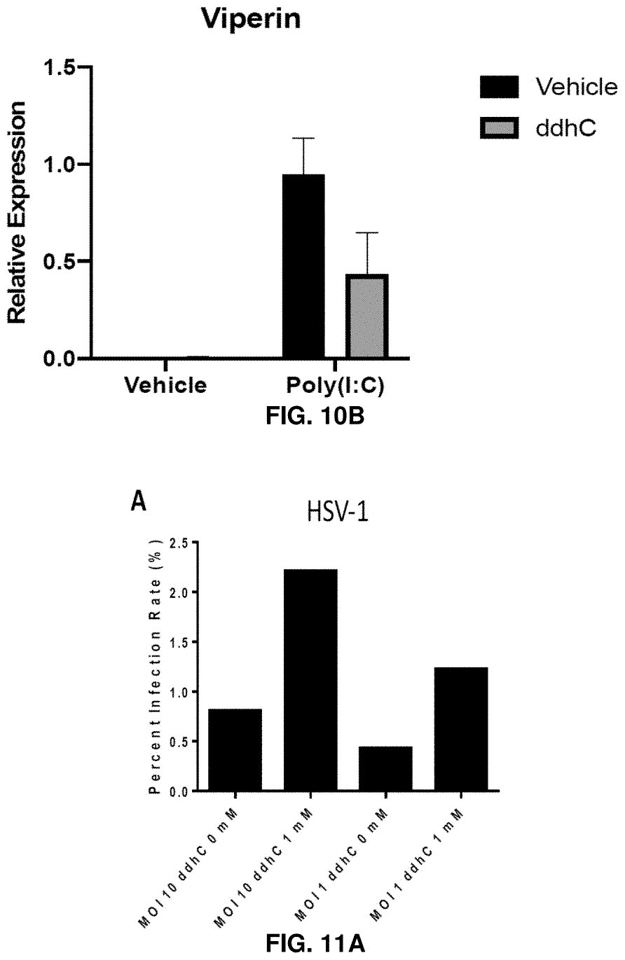

To establish a baseline for ddhCTP-mediated macrophage responses, murine bone marrow-derived macrophages (BMDMs) are stimulated with 1 mM ddhC or vehicle control (N=3 per condition). In these experiments, ddhC is imported by human cells and intracellularly phosphorylated to produce ddhCTP. Cells are harvested for RNA at 8 and 24 hours. Nanostring analysis revealed ddhC-treated cultures adopt expression profiles typically associated with anti-inflammatory phenotypes (FIG. 10A). To test whether ddhC can suppress inflammatory signaling in stimulated cells, BMDMs are treated with 1 mM ddhC or co-stimulated with 1 mM ddhC and 25 ug/mL Poly(I:C), a dsRNA viral mimic that stimulates antiviral immune responses (N=3 per condition). qPCR analysis reveals Poly(I:C)-stimulated BMDMs upregulate Viperin as part of the innate antiviral signaling response and this response is dampened when co-stimulated with ddhC (FIG. 10B).

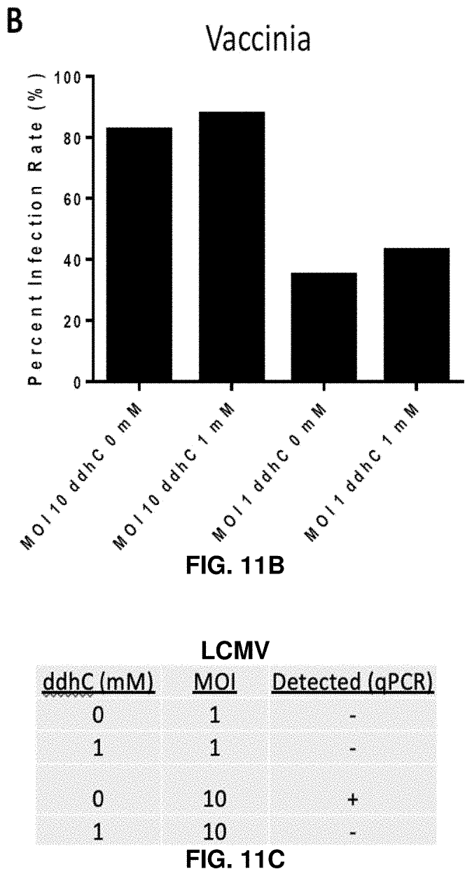

To determine whether ddhC is capable of suppressing viruses from non-flavivirus taxa, BMDMs are pre-treated with 1 mM ddhC. Next, cultures are infected with LCMV, HSV-1, or Vaccinia for three hours at a MOI of either 1 or 10, after which cultures are washed and fed with fresh media (N=1 per condition). After 24 hours, cultures are collected for analysis. HSV-1 and Vaccinia viruses used in this study contain a GFP CDS and infection rate is determined via flow cytometry. LCMV viral RNA is detected using qPCR following established methods. Preliminary results suggest ddhC suppresses LCMV, a negative strand RNA virus, but increases replication of HSV-1, a dsDNA virus (FIG. 11A, 11B, 11C). These data suggest that ddhC is capable of suppressing RNA viruses from non-flavivirus taxa, but is contraindicated in the setting of DNA virus infection.

### EXAMPLE 3

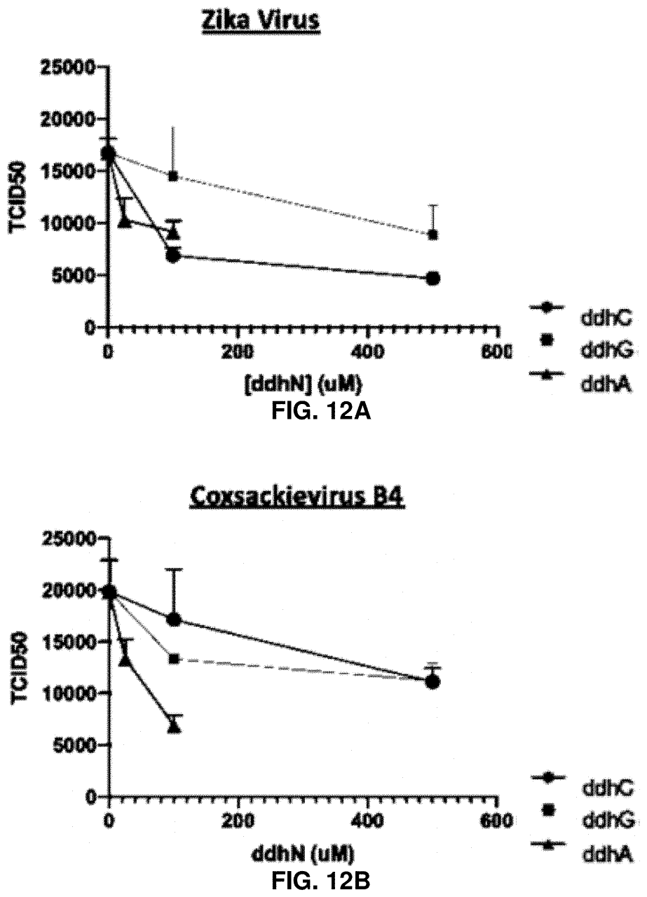

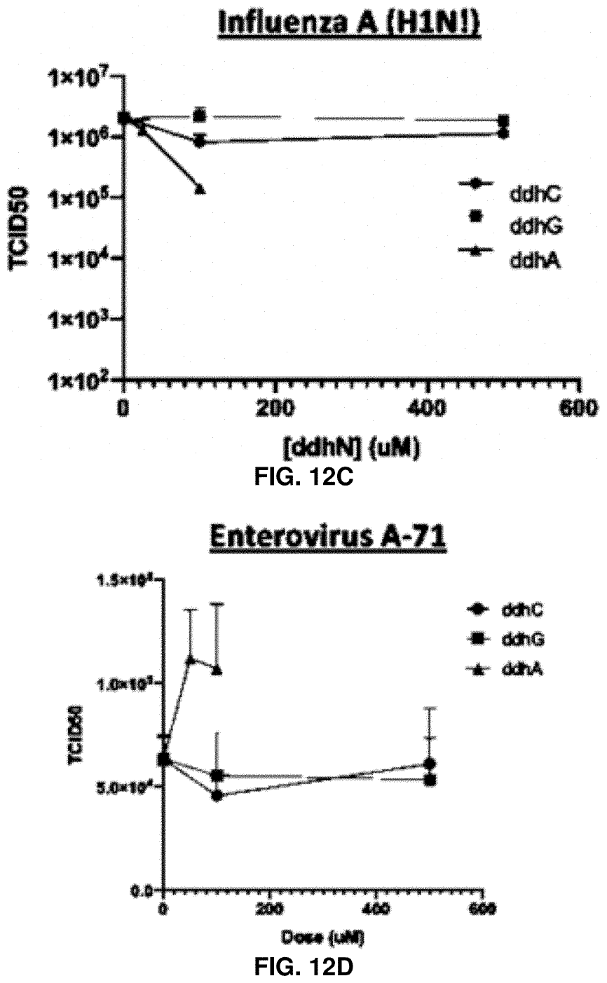

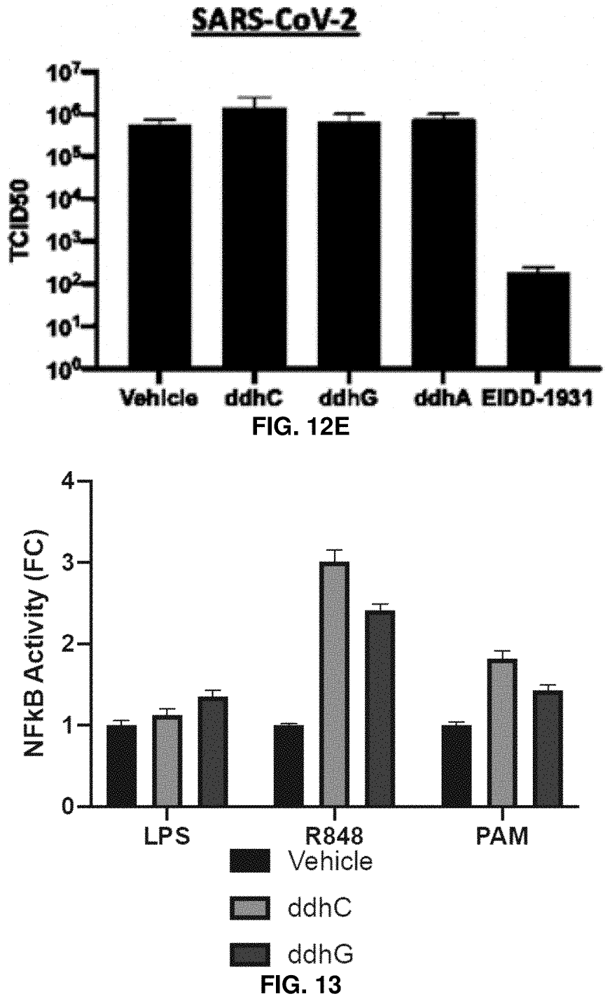

ddhNTPs are a novel class of antiviral small molecules. To date, the only mammalian virus known susceptible to ddhCTP antiviral activity is Zika Virus. Additionally, ddhC is the only member of the ddhN compound family previously tested against mammalian viruses. To determine the broader antiviral activity of the ddhNTP compound family, mammalian cells are pre-treated with ddhA, ddhC, or ddhG for 24 hours. Next cultures are infected with Zika Virus, Coxsackie B4 Virus, Influenza A, Enterovirus A-71, and SARS-CoV. Supernatant is then collected after at least 24 hours and viral titer is determined via TCID50 assay. The results for Zika Virus, Coxsackie B4 Virus, Influenza A, Enterovirus A-71, and SARS-CoV are shown in FIG. 12A to 12E, respectively. The data demonstrates that ddhNs effectively suppress Zika Virus, Coxsackie B4 Virus, and Influenza A

To determine the extent to which other ddhN derivatives also synergize with TLR agonists to enhance NFkB signaling, RAW264.7 cells containing an NFkB reporter are pre-treated overnight with either ddhC or ddhG and are subsequently stimulated with 500 ng/mL LPS, 1 μg/mL PAM, or 1 μg/mL R848. The results reveal that both ddhC and ddhG agonize TLR-mediated NFkB activation (FIG. 13).

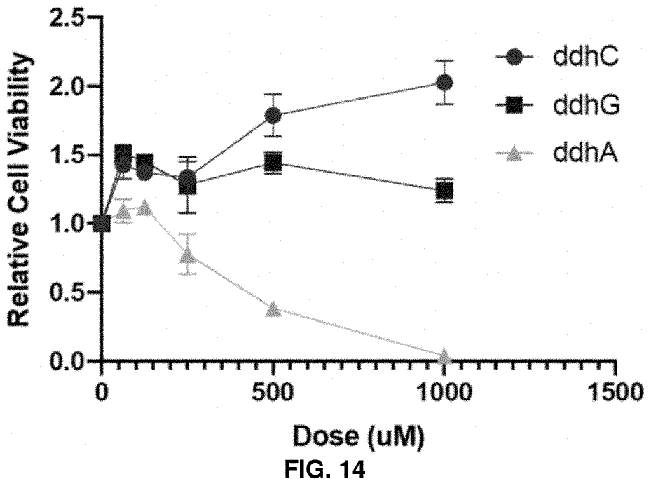

To determine whether ddhNTPs exhibit cytotoxic effects, Vero cells are treated overnight with increasing doses of ddhA, ddhC, or ddhG. After 24 hours, cell viability is determined via CCK8 per manufacturer's instructions. The results are shown in FIG. 14.

The following protein sequences produce the indicated ddhNs (Bernheim, Aude, et al. “Prokaryotic Viperins Produce Diverse Antiviral Molecules.” Nature, 2020). Also included is a sequence that produces a mixture of all three ddhUTP, ddhCTP, and ddhGTP from a single enzyme. This promiscuity suggests it may produce ddhATP, as well.

Using the Fibrobacter sequence indicated above a HMMER3 model is constructed that identifies conserved structural elements of the indicated Viperin homologs. This model reveals a highly conserved 4Fe-4S domain (Fer4_12 (PF13353.6)) and a Radical SAM superfamily domain Radical_SAM (PF04055.21). These domains are also observed when constructing HMMER3 models with the other sequences listed herein.

References cited:
Carissimo, Guillaume, et al. “Viperin Controls Chikungunya Virus—Specific Pathogenic T Cell IFNγ Th1 Stimulation in Mice.” Life Science Alliance, vol. 2, no. 1, 2019, doi:10.26508/lsa.201900298.
Chin, K.-C., and P. Cresswell. “Viperin (cig5), an IFN-Inducible Antiviral Protein Directly Induced by Human Cytomegalovirus.” Proceedings of the National Academy of Sciences, vol. 98, no. 26, 2001, pp. 15125-15130., doi:10.1073/pnas.011593298.

Gizzi, Anthony S., et al. “A Naturally Occurring Antiviral Ribonucleotide Encoded by the Human Genome.” Nature, vol. 558, no. 7711, 2018, pp. 610-614., doi:10.1038/s41586-018-0238-4.

Hinson, E. R., and P. Cresswell. “The Antiviral Protein, Viperin, Localizes to Lipid Droplets via Its N-Terminal Amphipathic a-Helix.” Proceedings of the National Academy of Sciences, vol. 106, no. 48, 2009, pp. 20452-20457., doi:10.1073/pnas.0911679106.
Hoek, Kylie H. Van Der, et al. “Viperin Is an Important Host Restriction Factor in Control of Zika Virus Infection.” Scientific Reports, vol. 7, no. 1, 2017, doi:10.1038/s41598-017-04138-1.
Jiang, D., et al. “Identification of Three Interferon-Inducible Cellular Enzymes That Inhibit the Replication of Hepatitis C Virus.” Journal of Virology, vol. 82, no. 4, 2007, pp. 1665-1678., doi:10.1128/jvi.02113-07.
Li, Wenli, et al. “The Antiviral Effect of Viperin for Type IV Dengue Virus Through the End of the C-Terminal.” Nanoscience and Nanotechnology Letters, vol. 11, no. 1, 2019, pp. 118-125., doi:10.1166/nn1.2019.2847.
Lynch, J. B., and E. Y. Hsiao. “Microbiomes as Sources of Emergent Host Phenotypes.” Science, vol. 365, no. 6460, 2019, pp. 1405-1409., doi:10.1126/science.aay0240.
Mimee, Mark, et al. “Programming a Human Commensal Bacterium, Bacteroides Thetaiotaomicron, to Sense and Respond to Stimuli in the Murine Gut Microbiota.” Cell Systems, vol. 1, no. 1, 2015, pp. 62-71., doi:10.1016/j.cels.2015.06.001.
Nasr, Najla, et al. “HIV-1 Infection of Human Macrophages Directly Induces Viperin Which Inhibits Viral Production.” Blood, vol. 120, no. 4, 2012, pp. 778-788., doi:10.1182/blood-2012-01-407395.

Shi, Na, et al. “Interaction between the Gut Microbiome and Mucosal Immune System.” Military Medical Research, vol. 4, no. 1, 2017, doi:10.1186/s40779-017-0122-9.
Szretter, K. J., et al. “The Interferon-Inducible Gene Viperin Restricts West Nile Virus Pathogenesis.” Journal of Virology, vol. 85, no. 22, 2011, pp. 11557-11566., doi:10.1128/jvi.05519-11.
Teng, Terk-Shin, et al. “Viperin Restricts Chikungunya Virus Replication and Pathology.” Journal of Clinical Investigation, vol. 122, no. 12, 2012, pp. 4447-4460., doi:10.1172/jci63120.
Villenave, Remi, et al. “Human Gut-On-A-Chip Supports Polarized Infection of Coxsackie B1 Virus In Vitro.” Plos One, vol. 12, no. 2, 2017, doi:10.1371/journal.pone.0169412.
Wang, Xiuyan, et al. “The Interferon-Inducible Protein Viperin Inhibits Influenza Virus Release by Perturbing Lipid Rafts.” Cell Host & Microbe, Cell Press, 15 Aug. 2007, www.sciencedirect.com/science/article/pii/S1931312807001357.
Wei, Chunyu, et al. “Viperin Inhibits Enterovirus A71 Replication by Interacting with Viral 2C Protein.” Viruses, vol. 11, no. 1, 2018, p. 13., doi:10.3390/v11010013.
Whitaker, Weston R., et al. “Tunable Expression Tools Enable Single-Cell Strain Distinction in the Gut Microbiome.” Cell, vol. 169, no. 3, 2017, doi:10.1016/j.cell.2017.03.041.
Zhou, Jie, et al. “Human Intestinal Tract Serves as an Alternative Infection Route for Middle East Respiratory Syndrome Coronavirus.” Science Advances, vol. 3, no. 11, 2017, doi:10.1126/sciadv.aao4966.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified host cell comprises a nucleic acid encoding a polypeptide, or set of polypeptides, having a biological activity of synthesizing an antiviral compound operatively linked to one or more promoters capable of expressing the polypeptide(s) in the genetically modified host cell.

2. The modified host cell of claim 1, wherein the genetically modified gut bacterial

3. The modified host cell of claim 2, wherein the genetically modified gut bacterial cell is a Bacteroides cell.

4. The modified host cell of claim 3, wherein the Bacteroides cell is a Bacteroides thetaiotaomicron, Bacteroides plebus or Bacteroides ovatus cell.

5. The modified host cell of claim 1, wherein the polypeptide has an amino acid sequence that has at least 70%, 80%, 90%, 95%, 99%, or 100% amino acid residue identity with one of SEQ ID NOs:1-5.

6. The modified host cell of claim 5, wherein the polypeptide comprises a conserved 4Fe-4S domain (Fer4_12 (PF13353.6)) and/or a Radical SAM superfamily domain Radical_SAM (PF04055.21).

7. The modified host cell of claim 6, wherein the conserved 4Fe-4S domain comprises conserved closely spaced cysteine residues in one of the following amino acid sequence:
CXXXCXXC (SEQ ID NO:6) or CNYXCXXC (SEQ ID NO:7),

8. The modified host cell of claim 1, wherein the antiviral compound is ddhATP, ddhCTP, ddhGTP, and/or ddhUTP.

9. The modified host cell of claim 5, wherein the polypeptide has an amino acid sequence that has at least 70%, 80%, 90%, 95%, 99%, or 100% amino acid residue identity with SEQ ID NO:1, and the conserved 4Fe-4S domain, such as CXXXCXXC (SEQ ID NO:6) or CNYXCXXC (SEQ ID NO:7), and the polypeptide has the biological activity of synthesizing ddhCTP.

10. The modified host cell of claim 5, wherein the polypeptide has an amino acid sequence that has at least 70%, 80%, 90%, 95%, 99%, or 100% amino acid residue identity with SEQ ID NO:2, and the conserved 4Fe-4S domain, such as CXXXCXXC (SEQ ID NO:6) or CNYXCXXC (SEQ ID NO:7), and the polypeptide has the biological activity of synthesizing ddhCTP.

11. The modified host cell of claim 5, wherein the polypeptide has an amino acid sequence that has at least 70%, 80%, 90%, 95%, 99%, or 100% amino acid residue identity with SEQ ID NO:3, and the conserved 4Fe-4S domain, such as CXXXCXXC (SEQ ID NO:6) or CNYXCXXC (SEQ ID NO:7), and the polypeptide has the biological activity of synthesizing ddhUTP.

12. The modified host cell of claim 5, wherein the polypeptide has an amino acid sequence that has at least 70%, 80%, 90%, 95%, 99%, or 100% amino acid residue identity with SEQ ID NO:4, and the conserved 4Fe-4S domain, such as CXXXCXXC (SEQ ID NO:6) or CNYXCXXC (SEQ ID NO:7), and the polypeptide has the biological activity of synthesizing ddhGTP.

13. The modified host cell of claim 5, wherein the polypeptide has an amino acid sequence that has at least 70%, 80%, 90%, 95%, 99%, or 100% amino acid residue identity with SEQ ID NO:5, and the conserved 4Fe-4S domain, such as CXXXCXXC (SEQ ID NO:6) or CNYXCXXC (SEQ ID NO:7), and the polypeptide has the biological activity of synthesizing ddhATP, ddhCTP, ddhGTP, and/or ddhUTP.

14. The modified host cell of claim 1, wherein the antiviral compound is ddhCTP, and the polypeptide(s) is Viperin, and optionally Cytidine/Uridine Monophosphate Kinase 2 (CMPK2).

15. The modified host cell of claim 14, wherein the Viperin is truncated to remove the N-terminal human localization signal.

16. The modified host cell of claim 15, wherein the polypeptide comprises a N-terminal tag that increases the expression and/or solubility of the polypeptide, such as MBP-GGGS-(E. coli), NusA-GGGS-(B. thetaiotaomicron), or SUMO-GGGS-(Saccharomyces cerevisiae).

17. The modified host cell of claim 1, wherein the genetically modified host cell is capable of colonizing an animal gastrointestinal (GI) tract. I

18. A composition comprising the genetically modified host cell of claim 1, and the antiviral compound, produced by the genetically modified host cell, in the composition but outside the genetically modified host cell; in that the antiviral compounds are produced by the genetically modified host cell and transported, moved, released or diffused to the outside of the genetically modified host cell.

19. A non-human animal comprising the genetically modified gut bacterial cell of claim 2 in the gastrointestinal (GI) tract of the non-human animal.

20. A method for making the genetically modified host cell, the method comprising: (a) optionally constructing a nucleic acid encoding a polypeptide, or set of polypeptides, having a biological activity of synthesizing an antiviral compound operatively linked to one or more promoters capable of expressing the polypeptide(s) in a genetically modified host cell, (b) introducing the nucleic acid into a host cell to generate the genetically modified host cell of claim 1, (c) optionally expressing the polypeptide(s) in order to produce the antiviral compound, and (d) optionally separating or isolating or purifying the antiviral compound from the genetically modified host cell.

21. A method for producing an antiviral compound in an animal, the method comprising: (a) optionally constructing a nucleic acid encoding a polypeptide, or set of polypeptides, having a biological activity of synthesizing an antiviral compound operatively linked to one or more promoters capable of expressing the polypeptide(s) in a genetically modified gut bacterial cell, (b) optionally introducing the nucleic acid into a gut bacterial cell to generate the genetically modified gut bacterial cell of claim 2, (c) introducing the genetically modified gut bacterial cell of the present invention into a gastrointestinal (GI) tract of an animal, and (d) producing the antiviral compound in the GI tract, wherein the genetically modified gut bacterial cell expresses the polypeptide(s) which in turn produces the antiviral compound in the genetically modified gut bacterial cell, such that the antiviral compound is transported, moved, released or diffused to the outside of the genetically modified gut bacterial cell into the GI tract.

22. A method for reducing the likelihood of a virus infection or reducing the severity or curing a virus infection in a subject, the method comprising: (a) administering a therapeutically sufficient number of a genetically modified gut bacterial cell of claim 2 to a subject in need of such treatment, wherein the likelihood of a virus infection in the subject is reduced, the severity of a virus infection is reduced in the subject or the subject is cured of a virus infection.
